(19) United States
(12) Patent Application Publication
(10) Pub. No.: US 2013/0158507 A1
(43) Pub. Date: Jun. 20, 2013

(54) Biliary System Catheter

(71) Applicant: Cook Medical Technologies LLC, Bloomington, IN (US)

(72) Inventor: Hilbert D. Brown, Winston-Salem, NC (US)

(73) Assignee: Cook Medical Technologies LLC, Bloomington, IN (US)

(21) Appl. No.: 13/713,221

(22) Filed: Dec. 13, 2012

Related U.S. Application Data
(60) Provisional application No. 61/577,942, filed on Dec. 20, 2011.

(51) Int. Cl. A61M 25/00

(52) U.S. Cl. CPC A61M 25/0028
USPC 604/506; 604/264

(57) ABSTRACT

A catheter system and a method for accessing the bile duct are provided. The catheter system includes an elongate shaft having a proximal portion and a distal portion. The catheter also includes a first lumen extending at least partially through the elongate shaft, a distal portion of the first lumen extending generally parallel to a longitudinal axis of the elongate shaft and a distal port operably connected to the first lumen. The catheter system also includes a second lumen extending at least partially through the elongate shaft and a side port operably connected to the second lumen, the side port positioned proximal to the distal port and operably connected to the second lumen. The second lumen includes an angled wall portion adjacent to the side port. A marker is included on the distal portion of the elongate shaft, the marker configured to indicate the position of the side port.

### RELATED APPLICATIONS

This application claims the benefit of U.S. Provisional Application No. 61/577,942, filed Dec. 20, 2011, which is incorporated by reference herein in its entirety.

### TECHNICAL FIELD

This invention generally relates to catheters and methods for accessing a vessel or duct within the body of a patient, and in particular to catheters and methods that may be used to access ducts of the biliary system.

### BACKGROUND

Minimally invasive medical procedures are performed in various vessels and ducts in the body. In some procedures, access to a vessel or duct may be needed to place prosthetic devices, to obtain a biopsy sample or to otherwise remove tissue material or other substances.

Minimally invasive medical procedures are frequently performed in the biliary system. Entry to the biliary system is through the duodenum into Ampulla of Vater leading to the branch for the pancreatic duct or the common bile duct (CBD) and the gall bladder. In most procedures, entry to the CBD is achieved by cannulation using an endoscopic retrograde cholangiopancreatography (ERCP) catheter to gain entry to the bile duct. Typically, a wireguide is preloaded within a lumen of the catheter. Once successful cannulation is performed, the physician may advance the wireguide further into the CBD.

Performing an ERCP procedure requires skill and precision to minimize the trauma to the pancreas and to reduce the risk of pancreatitis. Undue trauma to the entry site at the papilla, the CBD or the pancreatic duct may cause edema or worse trauma that may hinder pancreatic fluid drainage. Injury to the pancreatic duct can lead to post procedural pancreatitis, a very painful and potentially fatal illness. In addition, advancing a catheter/wireguide for entry into the bile duct can be very difficult due to the anatomy of the papilla, the CBD and the pancreatic duct.

There is a need for a catheter and a method that can reduce or prevent acute pancreatitis when minimally invasive medical procedures are preformed in the biliary system. What is needed is a catheter that can minimize the trauma to the biliary system by minimizing the need for electrocautery procedures, such as ERCP, to access the biliary duct and to reduce the time needed for the procedure.

### SUMMARY OF THE INVENTION

Accordingly, it is an object of the present invention to provide a method and a system having features that resolve or improve on one or more of the above-described drawbacks.

A catheter system and a method for accessing the bile duct are provided. The catheter system includes an elongate shaft having a proximal portion and a distal portion. The catheter also includes a first lumen extending at least partially through the elongate shaft, a distal portion of the first lumen extending generally parallel to a longitudinal axis of the elongate shaft and a distal port operably connected to the first lumen. The catheter system also includes a second lumen extending at least partially through the elongate shaft and a side port operably connected to the second lumen, the side port positioned proximal to the distal port and operably connected to the second lumen. The second lumen includes an angled wall portion adjacent to the side port. A marker is included on the distal portion of the elongate shaft, the marker configured to indicate the position of the side port.

### BRIEF DESCRIPTION OF THE DRAWINGS

FIG. 1A is a side view of an embodiment of a catheter according to the present invention;

FIG. 1B is a sectional view through the catheter shown in FIG. 1A;

FIG. 2 is sectional view of the catheter shown in FIG. 1A;

FIG. 3 is a side view of an alternative embodiment of a catheter according to the present invention;

FIG. 4 is a side view of an alternative embodiment of a catheter according to the present invention;

FIG. 5 is a side view of an alternative embodiment of a catheter according to the present invention;

FIG. 6 is a diagrammatic view of a catheter positioned at an opening to the biliary system;

FIG. 7 is a diagrammatic view of a catheter positioned within the pancreatic duct;

FIG. 8 is a diagrammatic view of a wire guide advancing through the a side port of the catheter into the bile duct; and

FIG. 9 is a diagrammatic view of the wire guide positioned in the bile duct.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the catheter to a patient. Hence the term “distal” means the portion of the catheter that is farthest from the physician and the term “proximal” means the portion of the catheter that is nearest to the physician.

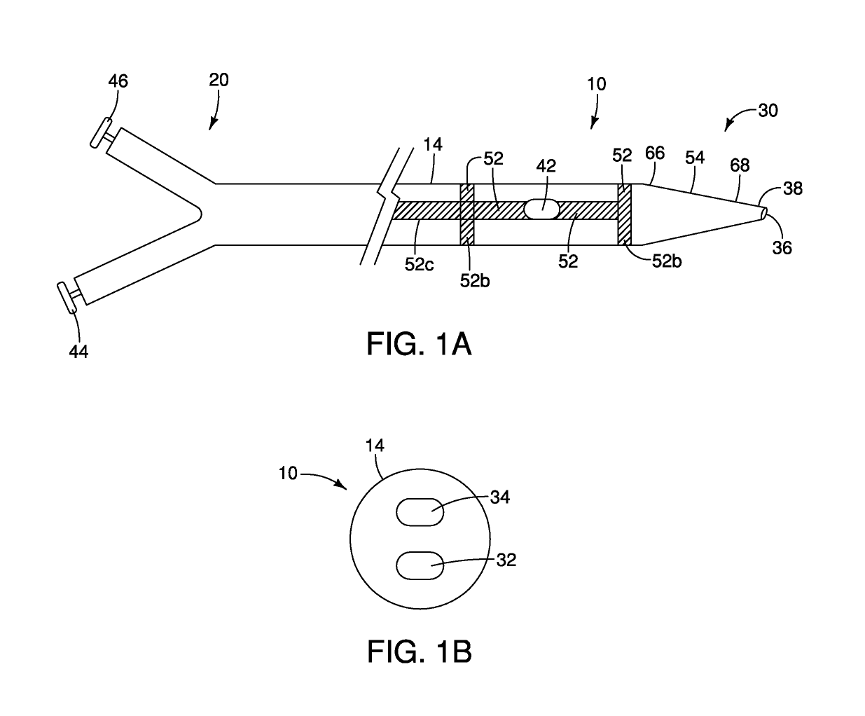

FIG. 1A illustrates a catheter 10 in accordance with an embodiment of the present invention. The catheter 10 includes an elongate body 14 having a proximal portion 20 and a distal portion 30. In some embodiments, the catheter 10 includes a first lumen 32 and a second lumen 34 as shown in the sectional view in FIG. 1B. In some embodiments, the first and second lumens 32, 34 extend from the proximal portion 20 to the distal portion 30. In some embodiments the first and second lumens 32, 34 may be configured for rapid exchange of devices extending through the lumens 32, 34. Additional lumens may also be included in the catheter 10.

The distal portion 30 of the catheter 10 includes a distal port 36 at a distal end 38 of the catheter 10. The distal port 36 is connected to the first lumen 32. The catheter 10 includes a side port 42 positioned proximal to the distal port 36. The side port 42 is connected to the second lumen 34. As shown in the embodiment in FIG. 1A, the proximal portion 20 may include a first proximal port 44 and a second proximal port 46. The first proximal port 44 may be operably connected to the first lumen 32 and the distal end port 36. The second proximal port 46 may be operably connected to the second lumen 34 and the side port 42. The first and second proximal ports 44, 46 may include a luer fitting and may be configured to receive a wire guide therethrough and may be configured for fluid delivery such as for contrast or saline and the like.

The distal end portion 30 of the catheter 10 may also include one or more markings 52. In some embodiments, the markings 52 may be used for alignment of the side port 42 with the bile duct. As shown in FIG. 1A, the markings 52 may include a longitudinal marking 52a longitudinally extending across the side port 42 to indicate the position of the side port 42 on the circumference on the body 14. The markings 52 may also include a pair of laterally extending markings 52b positioned on either side of the side port 42 and extending generally perpendicular to the longitudinal marking 52a to indicate the position of the side port 42 along the length of the body 14. The laterally extending markings 52b may extend around a circumference of the body 14 or partially across the longitudinal marking 52a. An additional longitudinal marking 52c may be included to determine the orientation of the side port 42 relative to the circumference of the body 14 (for example, at the 11 o'clock position) and to the bile duct. Other types of markings may also be included, for example, angular markings to help show movement of the catheter 10.

The distal end portion 30 of the catheter 10 may also include a tapered tip portion 54. The tapered tip portion 54 angles inward from the body 14 to the distal tip 38 and begins tapering distal to the side port 42. The tapered portion 54 may taper from a first diameter beginning at a proximal taper portion 66 to a second, smaller diameter at a distal taper portion 68. By way of non-limiting example, the tapered portion 54 may taper from about 7 French (Fr) (2.3 mm, 0.092 inches) at the proximal taper portion 66 to about 5 Fr (1.67 mm, 0.066 inches) at the distal taper portion 68. In some embodiments, the tapered portion 54 may be symmetrically tapered as shown for example in FIG. 1A. In some embodiments, the tapered portion 68 may be asymmetrically tapered as shown in FIG. 4. In some embodiments, the tapered portion is between about 1-3 inches (about 2.5-7.6 cm). In some embodiments, the tapered portion is about 2 inches (about 5.1 cm). Other sizes for the tapered portion 54 are also possible. The distal taper portion 68 may be sized and shaped to insert at least partially into the pancreatic duct as described in more detail below.

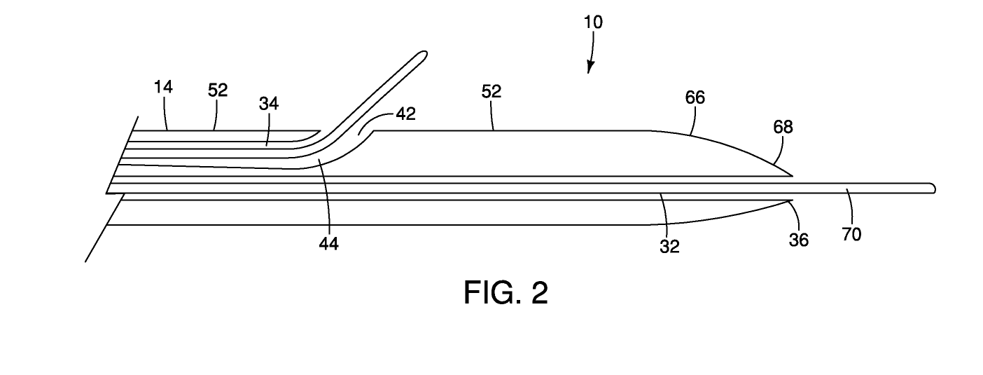

FIG. 2 illustrates a sectional view of an embodiment of the catheter 10. A first wire guide 70 is shown extending through the first lumen 32 and out of the distal port 36 at the distal end 38. In some embodiments, the first lumen 32 and first wire guide 70 extend distally along a longitudinal axis of the body 14. A second wire guide 72 is shown extending through the second lumen 34 and out of the side port 42. A distal wall portion 44 of the lumen 34 may be angled toward the side port 42 so that the second wire guide 72 exits the side port 42 at an angle with respect to the longitudinal axis of the body 14. In some embodiments, the angle that the second wire guide 72 exits the side port 42 is less than about 90°. In some embodiments, the side port 42 may be a skived opening in the catheter 10.

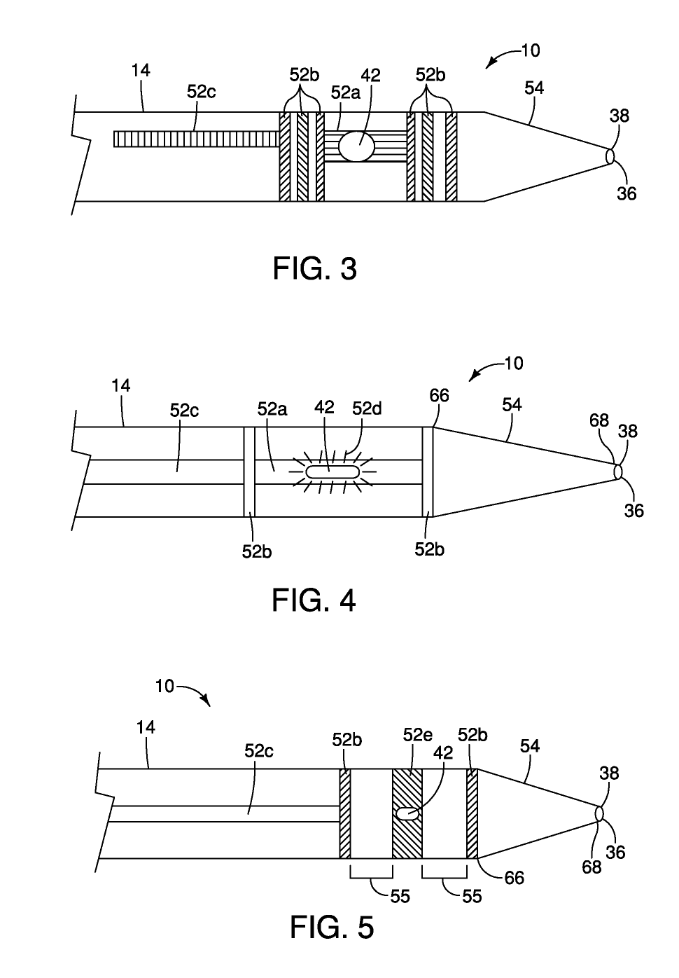

FIG. 3 illustrates an embodiment of the catheter 10 showing a different patterns of markings 52 than illustrated in FIG. 1A. The markings 52 may include the longitudinal marking 52a longitudinally extending across the side port 42. The markings 52 may also include a plurality of laterally extending markings 52b positioned on either side of the side port 52 and extending generally perpendicular to the longitudinal marking 52a. The plurality of laterally extending markings 52b may extend around a circumference of the body 14 or partially across the longitudinal marking 52a. The plurality of laterally extending markings 52b may have a different pattern on each marking to help determine the proximity of the side port 42 to the bile duct and to see movement of the catheter 10. An additional longitudinal marking 52c may be included to determine the orientation of the side port 42 relative to the circumference of the body 14 and to the bile duct and to align the side port 42 with the bile duct opening. In some embodiments, the marking 52c is positioned between the 11 o'clock position and the 1 o'clock position on the body 14 of the catheter 10 to facilitate alignment of the side port 42 with the bile duct opening.

FIG. 4 illustrates an alternative embodiment of the catheter 10 showing another pattern of markings 52. The side port 42 may include markings 52d that surround the side port 42 and are different from the longitudinally extending marking 52a to further facilitate positioning the catheter 10 at the proper position within the biliary system. In some embodiments, the distal laterally extending marking 52b may be positioned to indicate the proximal portion 66 of the tapered portion 54. (See for example, FIGS. 4 and 5.)

FIG. 5 illustrates an alternative embodiment of the catheter 10 showing another pattern of markings 52. The catheter 10 may include a plurality of laterally extending markings 52b positioned on the distal side and the proximal side of the side port 42 similar to the embodiments described above. The catheter 10 may also include a laterally extending marking 52e that extends across the side port 42. The marking 52e may extend around the circumference of the body 14 or partially around the circumference of the body 14. In some embodiments, the laterally extending markings 52b may be spaced equidistant from the marking 52e at a distance 55 apart. The catheter 10 shown in FIG. 5 may also include a longitudinally extending marker 52c that is aligned with the side port 42 to determine the position of the side port 42 relative to the circumference of the body 14 and to the bile duct and to align the side port 42 with the bile duct opening. The distal laterally extending marking 52b may be positioned to indicate the proximal portion 66 of the tapered portion 54. Other types of markings may also be included, for example, angular markings to help show movement of the catheter 10.

The catheter 10 may be of any size having a distal tip portion suitable for insertion into the papilla of the biliary system. In some embodiments, the body 14 of the catheter 10 may have a diameter from about 5 Fr to about 10 Fr with the tapered tip 54 tapering to about 5 Fr to about 3 Fr depending on the size of the papilla. The catheter 10 may be made from any material known to one skilled in the art suitable for use in the biliary system. The tapered portion 54 may be made from any material, for example, but not limited to nylon and urethane. In some embodiments, the catheter 10 may be formed from a material to facilitate smooth advancement into the biliary system.

In some embodiments, the catheter 10 may include radiopaque markings 52 to help with placement of the catheter 10 in the biliary system and for placement of the second wire guide 72 in the bile duct. In some embodiments, the catheter 10 may include markings 52 created by a laser or ink so the catheter 10 may be visualized using fluoroscopy, x-ray or ultrasound. Any type of visualization marking known to one skilled in the art may be used with the catheter 10.

The first and second wire guides may be any type of wire guide known to one of skill in the art. By way of non-limiting example, the wire guide may be a 0.035 inch (0.889 mm) diameter wire guide. Additional wire guide diameters may also be used and should be sided for advancement into the biliary system. The first and second wire guides may also include markings for visualization of the position of the wire guides during positioning within the biliary system. Suitable markings include any type of marking viewable using fluoroscopy, x-ray, ultrasound or other visualization technique. In some embodiments, the tips of the wire guides may be radiopaque to facilitate assessment of the position of the wire guides within the pancreatic duct and the bile duct.

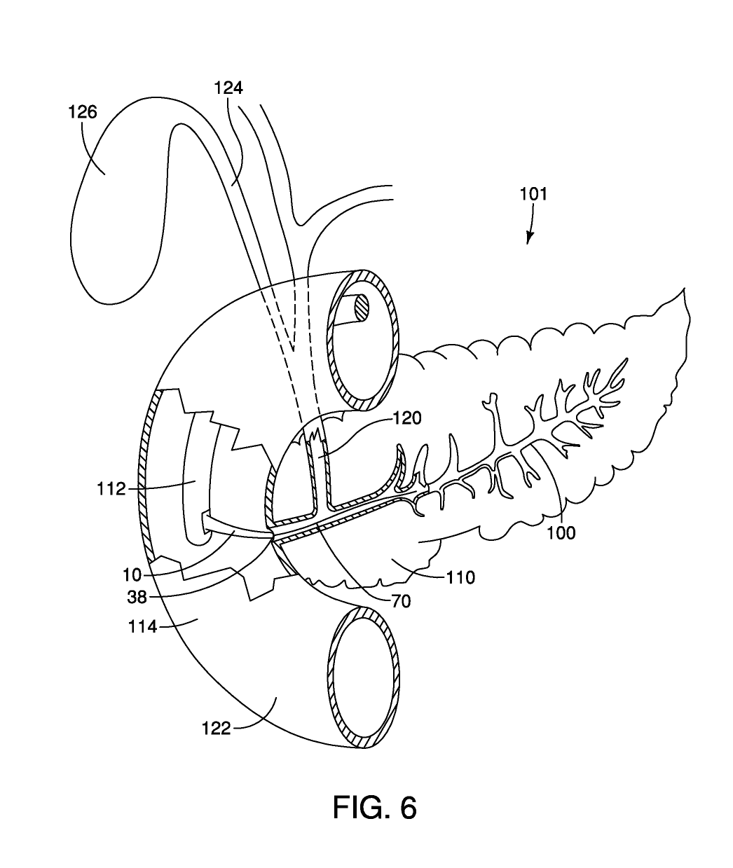

An exemplary method of delivering a wire guide to the biliary duct using the catheter 10 of the present invention without the need for precutting to enlarge the access to the biliary duct is illustrated in FIGS. 6-9. FIG. 6 illustrates the biliary system 101 including pancreatic duct 100 of the pancreas 110 and the bile duct 120. For reference, the duodenum 122, the cystic duct 124 and the gall bladder 126 are also shown. As shown in FIG. 6, the catheter 10 is delivered to the biliary system 101 using an endoscope 112. The endoscope 112 is delivered to the duodenum 122 near an opening to the Ampulla of Vater 114. The distal tip 38 of the catheter 10 is advanced from the endoscope 112 and positioned adjacent the opening of the papilla 114 of the biliary system 101. The first wire guide 70 is advanced distally through the distal tip 38 of the catheter 10 and into the pancreatic duct 100 of the pancreas 110.

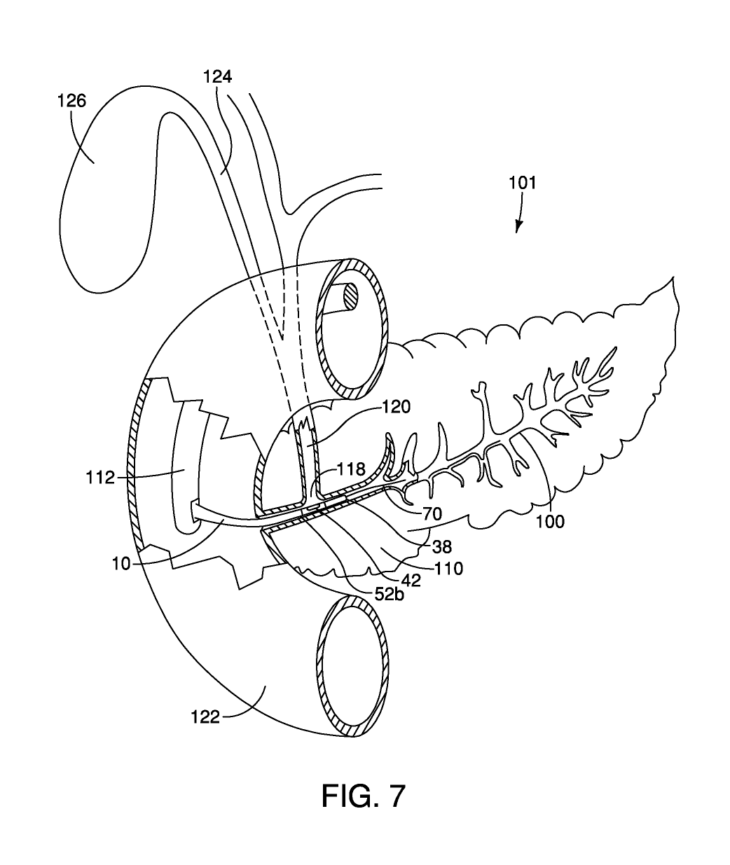

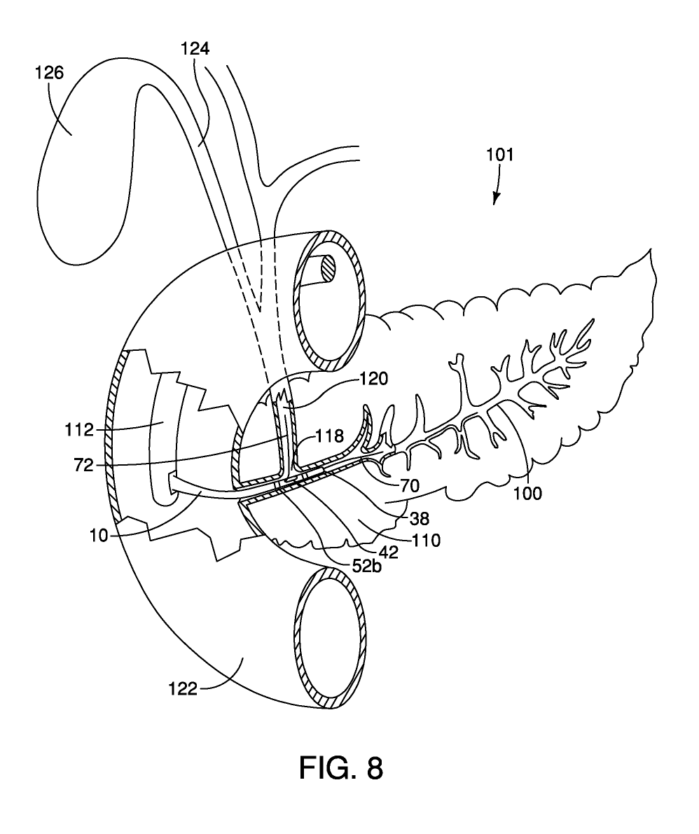

As shown in FIG. 7, the distal tip 38 of the catheter 10 is advanced distally along the wire guide 70 into the pancreatic duct 100. Advancement of the catheter 10 may be monitored by radiography, fluoroscopy, ultrasound or any other technique known to one skilled in the art and depending on the type(s) of markings 52 on the catheter 10. The catheter 10 may be distally advanced into the pancreatic duct 100 until the markings 52 indicate that the side port 42 of the catheter 10 is aligned near the opening 118 to the bile duct 120. The markings 52 allow for the side port 42 to be aligned both longitudinally and rotationally within the duct so that the second wire guide 72 can be extended into the bile duct 120. The distal wall portion 44 of the lumen 42 of the catheter 10 (see FIG. 2) facilitates positioning of the wire guide 72 in the bile duct 120 by changing and angling the direction of the wire guide 72 as the second wire guide 72 advances distally and exits the side port 42. The position of second wire guide 72 exiting the side port 42 can be monitored to ensure delivery of the second wire guide 72 to the bile duct 120 and then the second wire guide 72 may be advanced further into the bile duct 120. FIG. 8 illustrates the second wire guide 72 exiting the side port 42 of the catheter 10 and extending into the bile duct 120 without the need for electrocautery of the papilla to access the bile duct 120.

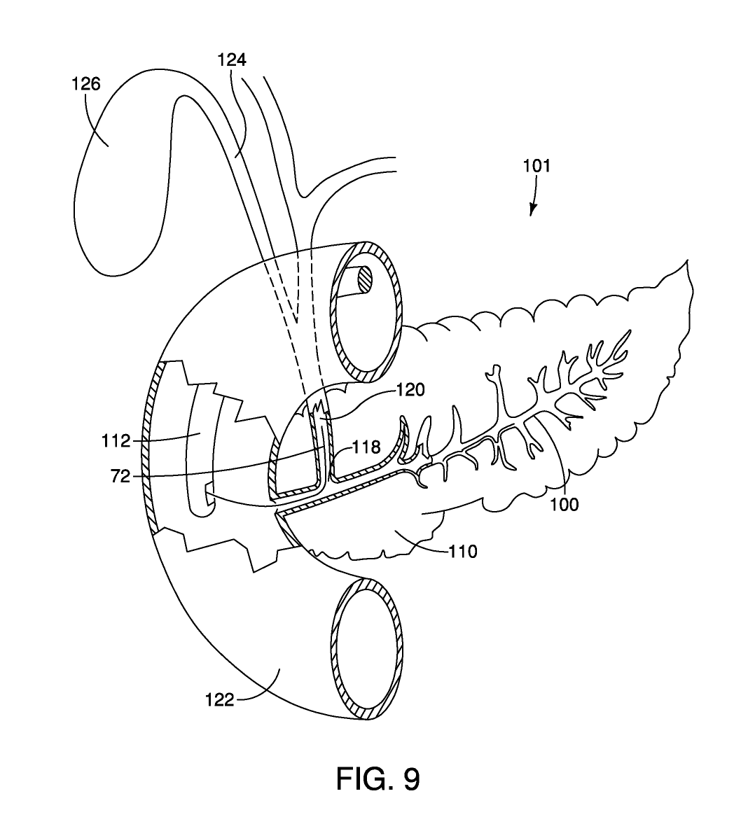

Once the second wire guide 72 is positioned within the bile duct 120, the first wire guide 70 and the catheter 10 may be withdrawn, leaving the second wire guide 72 positioned in the bile duct 120 as shown in FIG. 9. Additional medical devices may be delivered to the bile duct 120 over the second wire guide 72 as needed depending on the procedure. The first wire guide 70 may also remain in the pancreatic duct 100 and the catheter 10 withdrawn if any procedures are to be performed in the pancreatic duct 100. Medical devices may be delivered over the first wire guide 70 to the pancreatic duct 100.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. For example, the invention has been described in the context of the biliary system for illustrative purposes only. Application of the principles of the invention to any other bifurcated lumens or vessels within the body of a patient, including areas within the digestive tract, as well as areas outside the digestive tract such as other vascular systems, by way of non-limiting examples, are within the ordinary skill in the art and are intended to be encompassed within the scope of the attached claims.

1. A catheter system for accessing the bile duct, the catheter system comprising:
a catheter comprising:
an elongate shaft having a proximal portion and a distal portion,
a first lumen extending at least partially through the elongate shaft, a distal portion of the first lumen extending generally parallel to a longitudinal axis of the elongate shaft;
a distal port operably connected to the first lumen, the distal port positioned at a distal end of the elongate shaft;
a second lumen extending at least partially through the elongate shaft;
a side port operably connected to the second lumen, the side port positioned proximal to the distal port and operably connected to the second lumen, the second lumen comprising an angled wall portion adjacent to the side port; and
a marker on the distal portion of the elongate shaft, the marker configured to indicate the position of the side port.

2. The catheter system of claim 1, wherein the elongate shaft further comprises a tapered tip portion.

3. The catheter system of claim 2, wherein the tapered tip portion is distal to the side port and extends to the distal end.

4. The catheter system of claim 2, wherein the tapered tip portion tapers from about 7 Fr to 5 Fr.

5. The catheter system of claim 2, wherein the tapered tip portion is about 2 inches (about 5.1 cm) in length.

6. The catheter system of claim 2, where in the tapered tip portion is asymmetrically tapered.

7. The catheter system of claim 1, wherein the marker comprises a longitudinally extending marking extending across the side port.

8. The catheter system of claim 1, wherein the marker comprises a plurality of lateral markings extending substantially perpendicular to the longitudinal axis, wherein at least one of the plurality of lateral markings is positioned distal to the side port and at least one of the plurality of lateral markings is positioned proximal to the side port.

9. The catheter system of claim 1, wherein the marker comprises a longitudinally extending marking to indicate the circumferential position of the side port.

10. The catheter system of claim 1, wherein the marker comprises a laterally extending marking extending across the side port to indicate the position of the side port.

11. The catheter system of claim 1, wherein the marker comprises a laterally extending marking indicating a beginning of a proximal taper portion to a tapered tip portion.

12. The catheter system of claim 1, wherein an angle of the angled wall portion is less than about 90°.

13. The catheter system of claim 1, wherein the tapered portion comprises nylon or urethane or nylon and urethane.

14. The catheter system of claim 1, wherein the catheter system further comprises a first wire guide and a second wire guide.

15. A method of positioning a wire guide in a bile duct, the method comprising:
delivering a catheter to a bodily lumen and positioning a tip of the catheter adjacent to a papilla at an entry to a pancreatic duct and the bile duct;
distally extending a first wire guide out of a distal end port of the catheter, through the papilla and into the pancreatic duct;
distally advancing the catheter over the first wire guide and extending a distal tapered portion of the tip of the catheter into the pancreatic duct;
positioning a side port of the catheter at an entry to the bile duct;
advancing a second wire guide distally through the side port and into the bile duct.

16. The method of claim 15, comprising radiographically viewing the markings on the catheter to facilitate positioning of the side port at the bile duct.

17. The method of claim 15, comprising viewing a first marking to longitudinally position the side port of the catheter relative to the bile duct.

18. The method of claim 17, comprising viewing a second marking to rotationally position the side of port of the catheter relative to the bile duct.

19. The method of claim 15, wherein the second wire guide is advanced at an angle from a lumen of the catheter through the side port to position the second wire guide in the bile duct.

20. The method of claim 15, comprising withdrawing the catheter and leaving the second wire guide in position in the bile duct.
